(19) United States
(12) Patent Application Publication
(10) Pub. No.: US 2012/0258519 A1
(43) Pub. Date: Oct. 11, 2012

(54) Protein Harvesting

(75) Inventor: Sarfaraz K. Niazi, Deerfield, IL (US)

(73) Assignee: Therapeutic Proteins Inc., Chicago, IL (US)

(21) Appl. No.: 13/083,589

(22) Filed: Apr. 10, 2011

(51) Int. Cl. C12N 9/24; C07K 14/575; C07K 14/765; C12N 9/90; C07K 14/52; C07K 14/78; C12N 9/74; C07K 16/00; C07K 14/00; C07K 1/16; C07K 1/14; C12N 9/26

(52) U.S. Cl. 435/200; 530/412; 530/399; 530/364; 435/233; 435/201; 530/356; 530/382; 435/214; 530/387.3; 530/381; 530/395

(57) ABSTRACT

Methods of harvesting proteins directly from bioreactors to avoid at several steps in the purification of recombinant drugs are disclosed.

### FIELD OF THE INVENTION

The instant invention relates to a novel method of harvesting and purifying proteins.

### BACKGROUND

The present invention relates to a novel process for harvesting a recombinant protein, which process is characterized by that it comprises a combined step of adsorbing proteins in solution on a resin, removing the resin from the bioreactor and releasing the protein from resin-protein complex, allowing for a more rapid and efficient process of protein separation from culture media than previously provided by any separation process available within the field of the art.

Downstream processing involves cleaning up crude proteins to yield high purity products. Traditionally, these involve use of chromatography columns and highly specialized media to capture and purify the desired proteins. With an exponential rise in the number of protein drugs being developed and marketed, there have been remarkable developments in the field of downstream processing. Still, the time and cost-consuming steps of filtration, chromatography and purification slow down the manufacturing process and add substantial capital cost requirement to establish cGMP-grade manufacturing operations. Recent surveys show that most biopharmaceutical companies consider downstream processing to be their biggest concern since the upstream processing efficiencies have improved creating an imbalance in synchronizing the processes. (7th Annual Report and Survey of Biopharmaceutical Manufacturing. BioPlan Associates, Inc; Rockville, Md.).

The improvements in the downstream processing are mostly focused on creating better resins such as Protein A or more specific antibodies and converting existing systems into disposable forms, often streamlining the various upstream and downstream processing. Given below is a summary of the art that has been developed or under development:
Single-use downstream chromatography: Novozymes's new patented Dual Affinity Polypeptide technology platform replaces Protein A process steps with similar, but disposable, technology
Stimuli responsive polymers enable complexation and manipulation of proteins and allow for control of polymer and protein complex solubility, which results in the direct capture of the product without centrifuges or Protein A media, from Millipore Corp
Mixed mode sorbents to replace traditional Protein A and ion exchange, for improved selectivity and capacity with shorter residence times. These media, with novel chemistries, include hydrophobic charge induction chromatography, such as MEP, and Q and S HyperCel from Pall Corp
Monoliths, involving chromatography medium as a single-piece homogeneous column, such as Convective Interaction Media monolithic columns from BIA Separations
Simulated moving beds, involving multicolumn countercurrent chromatography, such as BioSMB from Tarpon Biosystems
Protein G (multiple vendors)
Single domain camel-derived (camelid) antibodies to IgG, such as CaptureSelect from BAC
New inorganic ligands, including synthetic dyes, such as Mabsorbent A1P and A2P from Prometic Biosciences
Expanded bed adsorption chromatography systems, such as the Rhobust platform from Upfront Chromatography
Ultra-durable zirconia oxide-bound affinity ligand chromatography media from ZirChrom Separations
Fc-receptor mimetic ligand from Tecnoge
ADSEPT (ADvanced SEParation Technology) from Nysa Membrane Technologies
Membrane affinity purification system from PurePharm Technologies
Custom-designed peptidic ligands for affinity chromatography from Prometic Biosciences, Dyax, and others
Protein A- and G-coated magnetic beads, such as from Invitrogen/Dynal
New affinity purification methods based on expression of proteins or MAbs as fusion proteins with removable portion (tag) having affinity for chromatography media, such as histidine) tags licensed by Roche (Genentech)
Protein A alternatives in development, including reverse micelles (liposomes), liquid-liquid extraction systems, crystallization, immobilized metal affinity chromatography, and novel membrane chromatography systems
Plug-and-play solutions with disposable components (e.g., ReadyToProcess), process development ÄKTA with design of experiments capability, and multicolumn continuous capture, from GE Healthcare.
Affinity Chromatography is a separation technique based upon molecular conformation, which frequently utilizes application specific resins. These resins have ligands attached to their surfaces, which are specific for the compounds to be separated. Most frequently, these ligands function in a fashion similar to that of antibody-antigen interactions. This “lock and key” fit between the ligand and its target compound makes it highly specific.
Lectin Chromatography. Many membrane proteins are glycoproteins and can be purified by lectin affinity chromatography. Detergent-solubilized proteins can be allowed to bind to a chromatography resin that has been modified to have a covalently attached lectin.
Immunoaffinity chromatography resin employs the specific binding of an antibody to the target protein to selectively purify the protein. The procedure involves immobilizing an antibody to a column material, which then selectively binds the protein, while everything else flows through.

It is surprising that most of the innovations listed above and those that form prior art involved selective interaction between a target protein and a binding material such as a resin to purify the protein to the limits of pharmacopoeia which currently require a purity greater than 98% (European Pharmacopoeia) and most of manufacturers use an internal control limit of greater than 99.5% purity. However, prior to the commencement of the downstream purification process, the culture media needs to be treated to separate the target protein in its crude form as it is not possible to load purification columns with culture media without adversely affecting the separation characteristics of these columns and also without excessively prolonging the process of downstream processing that adversely affects the stability of the target protein, besides adding extremely large cost of using large columns, pumps and expensive resin.

There remains a large unmet need to develop a process wherein the target protein is non-selectively removed from the culture media or a refolding solution prior to subjecting it to customary purification processes. The instant invention is targeted to modify the existing methods of performing protein harvesting or protein capturing prior to purification chromatography to increase the throughput of manufacturing processing without adding expensive and technically challenging modifications.

The recombinant protein manufacturing involves growing genetically modified organisms or cells in a culture media, harvesting the target protein from the rest of the contents of the culture media including recombinant cells or organisms and then purifying the target protein using column chromatography.

In a first embodiment, the instant invention simplifies the harvesting of proteins in a bioreactor. The instant invention employs conditions, which in one step combines several in strong contrast to common state of the art of protein separation today, and involves a process with only a fewer steps. The method of the present invention presents a novel procedural step for simultaneously extracting and concentrating a protein of interest from a host cell, at the same time removing practically all, or at least the majority of the host cell proteins.

In particular, the present invention relates to a process for harvesting a recombinant protein, wherein a culture media containing host cells and target protein are subjected to a non-specific treatment with an inexpensive resin or a combination of resins that would bind all or substantially all of charged or uncharged molecular species, removing the resin-protein complex by simpler processes like decanting or passing through a large pore filter to discard the eluate that would contain host cells; the process may also involve introducing the resin in a pouch, which can then be simply extracted out allowing discarding of the rest of material in a bioreactor. The present invention thus obviates a major hurdle in the harvesting of proteins that involves filtering out host cells using a fine filter, not larger than 5μ, to retain host cells such as Chines Hamster Ovary Cells. When large volume of media is used, this process takes a very long time, adds substantial cost of filters, pumps, containers and space management. This step is then generally followed by a concentrating step wherein the volume of culture media is reduced most to one-tenth its volume using a cross-flow or micro filtration process, which takes a very long time to complete and again adds substantial cost of equipment and manpower and in some instances causes degradation of target protein. The instant invention combines these two steps into one simple step. The argument that if it is the intent to harvest and concentrate protein from a complex mixture containing host cells, why would it not be more efficient to remove the protein from the mixture instead of removing other components that are present in much larger quantities. This is what would be considered a contrarian teaching. In the instant invention, those peculiar characteristics of target proteins are exploited to separate them from the rest of the mixture by a non-specific binding to a resin or a mixture of resins. Obviously, such non-specific capture of target proteins would also capture other components of the mixture and that only requires using a much larger quantity of resin or a specific type of resin that might have specific affinity for the target protein. The removal of protein-resin complex is a much simpler process than the removal of host cells or reduction in the volume of mixture; any mechanical process such as decanting, centrifugation or even filtration would work. It is noteworthy that the slowest of all processes would be filtration but even the a much larger pore size filter can be used and since the purpose is to collect the filtrate, not the eluate, the cost of substantially lower. Additional novel modifications of this step include introducing resin in a pouch made up of a filter material that would retain the resin inside the pouch requiring simple removal of the pouch or pouches to remove the target protein from the mixture.

In a second embodiment of the instant invention, it can be applied to concentration of proteins in stages other than the bioreactors. The steps of harvesting proteins are also involved in the concentration of protein after it has been refolded in a very dilute solution and whereas these solutions are of high purity and can be readily filtered, it is most frequently seen that the filtration of a refolded solution results in a substantial loss of protein due to degradation; the instant invention resolves this problem by removing all or substantially all of protein solution from the refolding solution, removing the buffers and reconstituting the protein eluted from the resin-protein complex for further purification.

In a third embodiment, the instant invention can be applied to separation of any protein solution including industrial production of proteins.

In a fourth embodiment, the instant invention avoids obviates the need for costly filtration processes for every type of manufacturing of proteins as in almost all instances a concentration step is involved.

In a fifth embodiment, the instant invention provides a means of continuously removing expressed protein from a culture media to enhance the level of expression that may be depressed because of the higher concentration of protein in the mixture.

In a sixth embodiment, the instant invention provides a means of continuously removing expressed protein from a culture media to reduce the toxicity of the expressed protein to host cells and thus prolonging the cycles of expression substantially increasing the yields of production. In a biological system, a particular protein is expressed only in a specific subcellular location, tissue or cell type, during a defined time period, and at a particular quantity level. This is the spatial, temporal, and quantitative expression. Recombinant protein expression often introduces a foreign protein in a host cell and expresses the protein at levels significantly higher than the physiological level of the protein in its native host and at the time the protein is not needed. The over-expressed recombinant protein will perform certain function in the host cell if the protein is expressed soluble and functional. The function of the expressed recombinant protein is often not needed by the host cell. In fact the function of the protein may be detrimental to the proliferation and differentiation of the host cell. The observed phenotypes of the host cells are slow growth rate and low cell density. In some cases, the recombinant protein causes death of the host cell. These phenomena are described as protein toxicity. These recombinant proteins are called toxic proteins.

Protein toxicity is a commonly observed phenomenon. All active proteins will perform certain functions. The host cells need all of these functions with few exceptions and therefore they interfere with cellular proliferation and differentiation. The appeared phenotype of the effects of these proteins to the host cells is their “toxicity”. It is estimated that about 80% of all soluble proteins have certain degree of toxicity to their hosts. About 10% of all proteins are highly toxic to host cells. The completely insoluble or dysfunctional proteins will not be toxic to the host cell, though they may drain the cellular energy to produce them when over-expressed. Protein over-expression creates metabolic burden for the host cell, but this burden is not toxicity to the cell. Some low solubility or partially functional proteins may still be toxic to the host. While the exposure of the host cell to protein being expressed is inevitable and is only optimized through codon usage, once the protein has been expressed, it would be prudent to transport it out of the cell as soon as possible and this diffusion reaction requires establishing a sink condition that is readily achieved if the expressed protein in the surroundings of the host cell is removed from the solution such as in the case of the instant invention by binding to a resin.

In a seventh embodiment, the instant invention provides a means of increasing the chemical stability of expressed protein by binding it to a resin as soon as it is expressed as the chemicals are always less stable in a solution form than in a solid form or in this case a complex form; this would substantially improve the yield of production. The very nature of the recombinant product makes it unstable. Instability of a recombinant protein can be separated into either physical instability issues or chemical instability issues. Physical instability can be related to such things as denaturation of the secondary and tertiary structure of the protein; adsorption of the protein onto interfaces or excipients; and aggregation and precipitation of the protein. In most biopharmaceutical processes, additives are used to improve the physical stability of a protein. The addition of salts can significantly decrease denaturation and aggregation by the selective binding of ions to the protein. Polyalcohols can also be used to stabilize the protein by selective solvation. Finally, surfactants are often used to prevent the adsorption of proteins at the surface, although there is a fine line between the amount of surfactant needed to prevent adsorption and the amount needed to denature a product. In addition, excipients are often used to prevent aggregation. Chemical instability of a protein product results in the formation of a new chemical entity by cleavage or by new bond formation. Examples of this type of instability would be deamidation, proteolysis and racemization. There are some more obvious choices to improve the chemical instability, such as modulation of pH, the use of low temperatures for storage and processing, and the addition of preservatives. In the process of recombinant manufacturing where proteins are secreted into media, there are two methods widely used. In one method of batch processing, the proteins are harvested at the end of the cycle that might be as long as several weeks of continuous expression; while many proteins would survive the 37 C environment for that length of time, many would degrade over period of time. By capturing the proteins through formation of resin-protein complex, the stability of and thus the yield of production can be increased since in the complex stage, the molecules are immobilized and thus less likely to decompose. While many proteins may decompose by adsorbing to various surfaces, the interaction between a resin and protein is of a different nature as evidenced by the use of resins in the purification of proteins whereby high degree of stability is maintained when eluting from a resin column. In another situation, where a perfusion system is used for the upstream production of recombinant proteins, a portion of culture media is replaced with fresh media and the media removed is filtered of host cells, reduced in volume and either stored at a lower temperature or processed with downstream processing. This technique also adds substantial cost to production in media and its handling; by passing the media through a column containing the resin, which can be replaced with fresh resin periodically, the expressed protein can be removed readily without affecting the total count of viable host cells; while the resin might also absorb some of the nutrients, these can be easily replaced in a fed-batch culture systems.

In an eight embodiment, the instant invention provides a means of substantially reducing the cost of recombinant drug manufacturing by eliminating some of the most costly and time consuming steps. The cost of using a non-specific resin is minimal as this can be used repeatedly unlike the resin used in the downstream purification where it must be replaced periodically as it loses its power to resolve the separation. Until the resin breaks down or is physically damaged, it can be used continuously and even when the efficiency of adsorption is reduced, it can be mixed with fresh resin to give it a very long useful life. The purification of biological therapeutics generally involves the use of cross flow filtration (tangential flow filtration), normal flow filtration (dead ended filtration) combined with chromatographic separations. Cross flow filtration and normal flow filtration retain matter through size exclusion and are complementary to chromatography's selectivity. For processes where volumes are large such as into thousands of liters, the cost of equipment for filtration is into hundreds of thousands of dollars with expensive filters all adding to a cost that represents a major fraction of the total cost of manufacturing of recombinant drugs.

In the night embodiment, the instant invention combines several steps of upstream and downstream; in the harvesting process, the resin-protein complex can be directly treated with buffers to begin the first stage of purification and where the resin is carefully and artfully selected, lead to high purity of a protein in one step. The resin-protein complex is ready for downstream processing without the need to load a column intended for downstream processing and this can save substantial time for loading; this prolonged delay can also be detrimental to the stability of target protein.

In the tenth embodiment, the instant invention offers to eliminate a very laborious and expensive step of first stage filtration or other means of separating the protein harvested; by using a pouch to contain the resin, all steps generally required to remove resin such as decanting, centrifugation (low speed), filtration (coarse) can be avoided altogether; the pouches can be stringed together so that these are simply removed by picking up the end of the string at one end. The pouches can also be then packed directly in a column for elution as if this were loose resin. Since the pouches containing the resin can be pre-washed to remove the resin of particle size smaller than the porosity of the filter that forms the pouch, the losses of bound protein to resin will be eliminated. The pouches can be washed and re-used, perhaps requiring a sterilization step if these are used during the bioreaction cycle, a chemical can achieve the sterilization similar to what is used in the sanitization of the chromatography column. This method of holding the resin in a pouch further reduces any loss of resin and saves additional costs.

In the eleventh embodiment, the instant invention allows to adjust the physicochemical characteristics of the culture media to achieve optimal binding of proteins with resin improving the yield.

In the twelfth embodiment, the instant invention allows for the use of a mixed-bed resin that may contain an ionic resin, a hydrophobic resin and an affinity resin all used together to optimize the efficiency of harvesting. It is well established that the use of ionic resins does not allow complete capture of proteins because of the logarithmic nature of ionization; a combination of resins used in the instant invention allows for a more complete recovery of target proteins. Since the purpose of reaction at the resin-protein complexation stage is to harvest and not purify the protein, the calculations like chromatography plates for purification are not important and neither is the particle size o the resin allowing use of the cheapest resin available; any lack of efficiency in capturing proteins can be readily adjusted by increasing the quantity of resin. The resin can be used repeatedly after washing of the proteins and sanitizing the resin.

In the thirteenth embodiment, the instant invention describes a novel method of protein purification wherein all those steps which are expensive and time consuming are obviated; the method of protein purification involves adding to a solution of protein ready for purification, a resin that would be the first resin in the process of purification. By adding the resin directly to the vat containing a culture medium, the steps of removing host cells (a filtration process), reducing the volume of culture medium (a cross-flow filtration process), feeding the purification column with concentrated solution (column loading) are all combined in a single, simple and most cost-effective step: the resin added to the vat containing the protein solution is poured into a purification column once the resin-protein conjugate has been formed. This method further provides the option of adding more resin to assure that substantially all of protein in solution has bound to protein; in customary methods, a fixed volume of resin is first packed in a column and then the concentrated solution of protein pumped into the column resulting in a mismatch between the amount of protein in the concentrated solution and the calculated binding capacity since theoretical calculations of binding are not always replicated on the column due to a multitude of factors. The overall impact of this embodiment is quantifiable in terms of the time it takes to make a protein ready for purification; as a general guideline, if a 2000 L batch of a recombinant production is ready for processing, it will take about 10-12 hours to filter it through a 0.22 micron filter to remove host cells such as Chinese Hamster Ovary Cells; this step would then be followed by a cross-flow filtration process that might take 12-24 hours to reduce the volume to 200-300 liters; this step is then followed by loading on the column, which may take another 6-8 hours depending on the size of the column used. While the batch is subjected to above processes, the target protein is under going degradation, both because of the effects of temperature as well as the strain exerted on proteins in the filtration process. The instant invention offers a solution to replace all of these steps with a single short step. The resin, contained in filter bags is placed inside the container containing the final volume and allowed to equilibrate with culture media, binding proteins.

In the fourteenth embodiment, the instant invention describes a method of keeping the resin binding the protein separate from the culture media inside a bioreactor and thus allowing separation of wasted culture media and cells by simply draining the bioreactor; this eliminates at least three steps in downstream processing, viz., filtration of culture broth to remove cells, cross-flow filtration to reduce the volume of broth and finally loading of protein solution onto a separation column.

The embodiments described above do not in any way comprises all embodiments that are possible using the instant invention and one with ordinary skills in the art would find many more applications specific to a complex process or even in those processes where such needs might not be immediately apparent.

Prior art on using resins to harvest recombinant proteins is non-existent; the U.S. Pat. No. 7,306,934 issued on 11 Dec. 2007 to Arora et al., teaches the use of porous solid ion exchange wafer for immobilizing biomolecules, said wafer comprising a combination of an biomolecule capture-resin containing a transition metal cation of +2 valence; it also teaches a separative bioreactor, comprising an anode and a cathode, a plurality of reaction chambers at least some being formed from a porous solid ion exchange wafers (above) having a combination of art biomolecule capture-resin and an ion-exchange resin and having a genetically engineered tagged biomolecule immobilized on said biomolecule capture resin, each of said porous solid ion exchange wafers being interleaved between a cation exchange membrane and an anion exchange membrane, and mechanism for supplying an electric potential between the anode and the cathode. The instant invention is significantly different from the separative bioreactor taught by Arora. First, the instant invention does not require use of electrodes, resins with a transition cation of +2 valence or immobilized metal ion affinity chromatography. The use of EDI (electrodeionization) and specific use of tags and limited nature of solvents to remove the captured proteins mainly enzymes makes this patent teachings distinctly different from the instant invention. Moreover, the Arora patent adds a hardware that adds to the cost of processing purification of proteins while the instant invention combines several processes into one without adding any new cost element.

### EXAMPLE 1

Genetically modified CHO cells were brought to confluence using OptiCHo media (In Vitrogen) and allowed to achieve a steady expression stage wherein the host cells began expressing erythropoietin. A small sample of the culture was removed (2 mL) filtered and tested for erythropoietin concentration using ELISA method to show the concentration of erythropoietin as 100 mcg/mL in a 120 mL culture media. Phenyl sepharose resin was used to harvest the protein by adding in an amount equal to about 2 ml of resin that had earlier been washed with a buffer of pH 7 to remove ethanol from as its storage medium. The flask was shaken for half hour and another sample (2 mL) of medium was removed and filtered and tested to show that 99% of erythropoietin was captured.

### EXAMPLE 2

Nylon mesh of 20 micron square opening, 14% open area, 34 micron thread diameter (mesh 478) was used to make a pouch of 2×2 inch size and filled with phenyl sepharose resin, 5 gram in each pouch, the pouch was sealed at all ends by heat and the pouch weighed out and placed in purified water and stirred; after 10 minutes, the pouch was dried and weight to show less than 5% loss in the resin. A single pouch was added to the flask containing media and expressing erythropoietin and showed more than 99% capture of erythropoietin in the solution.

### EXAMPLE 3

To test a perfusion model, a pouch made as show in Example 2 was added to a flask containing a culture media and host cell producing erythropoietin and replaced daily with a fresh pouch every day for a period of 30 days; the culture kept growing with high viability and robust expression without any need to replenish the culture with any nutrients.

### EXAMPLE 4

As demonstrated in Example 1, the resin used was the same resin as intended for the first stage of purification of the protein, erythropoietin. After the harvesting of protein as resin-protein conjugate, the conjugate was allowed to settle down and the bioreactor decanted and the slurry of the conjugate poured into a downstream purification process fitted with a 5μ filter at the eluate side of the column and the drug purified as usual. This allowed elimination of three steps in the processing: removal of host cells, reduction of media volume and loading of column for purification.

What is claimed is:

1. A method of harvesting a target protein from a liquid in a first container comprising:
A means of contacting said target protein with a resin capable of binding substantially all of said target protein to form a protein-resin conjugate;
A means of separating said protein-resin conjugate from said liquid;
A means of recovering said target protein from said protein-resin conjugate.

2. The method of harvesting a target protein of claim 1 wherein harvesting comprises collecting, concentrating or removing dissolved or undissolved proteins in said liquid.

3. The method of harvesting a target protein of claim 1 wherein said target protein is selected from the group consisting of small proteins, enamel matrix proteins, hormones, parathyroid hormones, growth hormones, gonadotropins, insulin, ACTH, prolactin, placental lactogen, melanocyte stimulating hormone, thyrotropin, calcitonin, enkephalin, angiotensin, cytokines human serum albumin, bovine serum albumin, ovalbumin, glucose isomerase, α-amylase, and endo-β-glucanase, growth hormone (GH), IGF-1, IGF-2, PTH, PGE₂, TGF-β, TGF-α, bEGF, EGF, PDGF-AB, PDGF-BB, osteoprotegerin (OPG), osteopontin (OP), FGF-1, FGF-2, thyroid hormone, BMP-2, BMP-3, BMP-4, BMP-6, BMP-7, VEGF, 1.25(OH).sub.2 vitamin D.sub.3, caclitonin, IFN-gamma, OCN (osteocalcin), ON (osteonectin), OP-1 (osteogenic protein-1), NGF, collagen, fibronectin, fibrinogen, thrombin, factor XIII, a recombinant protein, a recombinant antibody and a recombinant peptide.

4. The method of harvesting a target protein of claim 1 wherein said target protein is an industrial protein.

5. The method of harvesting a target protein of claim 1 wherein said target protein is a fusion protein.

6. The method of harvesting a target protein of claim 1 wherein said target protein is a tag protein.

7. The method of harvesting a target protein of claim 1 wherein said first container additionally contains a culture comprising bacteria, yeast, hybrodomas, baculoviruses, mammalian cells or plant cells.

8. The method of harvesting a target protein of claim 7 wherein said culture is genetically modified to express said target protein.

9. The method of harvesting a target protein of claim 7 wherein said culture is lysed.

10. The method of harvesting a target protein of claim 1 wherein said liquid comprises a culture medium, a protein refolding solution or an industrial process solution.

11. The method of harvesting a target protein of claim 1 wherein said first container comprises a bioreactor, a fermenter, a retaining vessel for protein refolding or a reaction vessel.

12. The method of harvesting a target protein of claim 1 wherein said first container is a disposable container.

13. The method of harvesting a target protein of claim 1 wherein said first container is a flexible container.

14. The method of harvesting a target protein of claim 1 wherein said target protein is harvested at the end of a bioreactor, fermenter, protein refolding or a reaction cycle.

15. The method of harvesting a target protein of claim 1 wherein said wherein said protein is harvested during a bioreactor, fermenter, protein refolding or a reaction cycle.

16. The method of harvesting a target protein of claim 15 wherein said liquid is replenished with nutrient media lost due to adsorption of nutritional elements of said media contents onto said resin.

17. The method of harvesting a target protein of claim 1 wherein said target protein is harvested as a part of a manufacturing process.

18. The method of harvesting a target protein of claim 15 wherein said target protein is harvested to reduce or prevent toxicity to said culture.

19. The method of harvesting a target protein of claim 1 wherein said target protein is harvested to optimize chemical and physical stability of said target protein in said liquid.

20. The method of harvesting a target protein of claim 1 wherein said means of contacting said resin with said target protein comprises adding said resin to said liquid in said first container and letting it stand for sufficient period of time and providing sufficient agitation of said liquid to form said protein-resin conjugate.

21. The method of harvesting a target protein of claim 1 wherein said liquid is modified using procedures comprising pH adjustment, ionic strength adjustment and polarity adjustment to optimize binding of said target protein to said resin.

22. The method of harvesting a target protein of claim 1 wherein said means of contacting said resin with said protein comprises passing said liquid through a second container external to said first container and containing said resin and additional means of retaining said resin inside said second container comprising a filter material of a pore size smaller than the average diameter of said resin.

23. The method of harvesting a target protein of claims 1 and 22 wherein said means of contacting said resin with said protein comprises recirculating said liquid from said second container into said first container.

24. The method of harvesting a target protein of claims 22 and 23 wherein said second container is a disposable container.

25. The method of harvesting a target protein of claims 22 and 23 wherein said second container is a flexible container.

26. The method of harvesting a target protein of claims 22 and 23 wherein said second container is a chromatography column.

27. The method of harvesting a target protein of claims 22 and 23 wherein said second container is and two-dimensional or three-dimensional plastic bag.

28. The method of harvesting a target protein of claims 22 and 23 wherein said second container is a hard-walled container.

29. The method of harvesting a target protein of claims 22 and 23 wherein said target protein is contacted with said resin in said second container intermittently or periodically.

30. The method of harvesting a target protein of claim 22 wherein said liquid is modified for its pH, ionic strength or polarity by means of adding to second container simultaneous to the entry of said liquid from said first container, appropriate quantity of chemicals to modify said pH, said ionic strength or said polarity of said liquid.

31. The method of harvesting a target protein of claims 22 and 23 wherein said pore size of filter material is between 10 to 300 microns.

32. The method of harvesting a target protein of claims 22 and 23 wherein said method comprises a plurality of said second containers arranged in parallel or in series

33. The method of harvesting a target protein of claim 1 wherein said resin comprises an ionic-exchange resin, a hydrophobic resin, an affinity resin or a mixture thereof.

34. The method of harvesting a target protein of claim 1 wherein said resin comprises a mix-bed resin.

35. The method of harvesting a target protein of claim 1 wherein said resin comprises a Protein-A or Protein-G resin.

36. The method of harvesting a target protein of claim 1 wherein said resin comprises a resin with specific affinity towards said target protein.

37. The method of harvesting a target protein of claim 1 wherein said resin is removed and replaced with a fresh resin of the same or a different type periodically.

38. The method of harvesting a target protein of claim 1 wherein said resin is re-used after washing.

39. The method of harvesting a target protein of claim 1 wherein said means of separating protein-resin conjugate from said medium comprises decanting, filtration or centrifugation.

40. The method of harvesting a target protein of claim 1 wherein said means of recovering said target protein from said protein-resin conjugate comprises washing said resin-protein conjugate with a buffer capable of breaking said protein-resin conjugate and collecting the washing as a concentrated solution of said target protein.

41. The method of harvesting a target protein of claim 40 wherein said concentrated solution is filtered with through a filter of pore size of said smaller than 5μ.

42. The method of harvesting a target protein of claims 22 wherein said means of recovering said target protein from said protein-resin conjugate comprises eluting said target protein from said second container using a buffer capable or breaking said protein-resin conjugate and filtering the eluate through a filter material of pore size smaller than the average size of said culture and collecting said eluate as a concentrated solution of said target protein.

43. The method of harvesting a target protein of claims 23 wherein said means of recovering said target protein from said protein-resin conjugate comprises removing said protein-resin conjugate from said second container and washing said protein-resin conjugate using a buffer capable or breaking said protein-resin conjugate and filtering through a filter material of pore size smaller than the average size of said culture and collecting a filtrate of a concentrated solution of said target protein.

44. The method of harvesting a target protein of claims 42 and 43 wherein a plurality of said buffers are used in a series of application and said eluate from each series of applications is pooled.

45. The method of harvesting a target protein of claims 42 and 43 wherein said pore size of said filter material is 0.22 to 5 microns.

46. The method of harvesting a target protein of claim 1 wherein said means of contacting said resin with said protein comprise enclosing said resin in a pouch made of a filter material and adding said pouch or a plurality of said pouches to said liquid containing said protein.

47. The method of harvesting a target protein of claim 46 wherein said pouch is a cylinder, pillow or a ball shaped.

48. The method of harvesting a target protein of claim 46 wherein said pouch has walls made of a filter material of porosity smaller in size compared to the average particle diameter of said resin.

49. The method of harvesting a target protein of claim 48 wherein said filter material is a nylon mesh, a cellulose fiber or a membrane.

50. The method of harvesting a target protein of claim 46 wherein said pouch is first soaked in water or a buffer solution to remove any particles of said resin smaller than said porosity of said filter.

51. The method of harvesting a target protein of claim 48 wherein said porosity comprises holes in said filter material of less than 20μ in diameter.

52. The method of harvesting a target protein of claim 46 wherein said pouch is connected with another said pouch by means of a string, clip or a hook.

53. The method of harvesting a target protein of claim 46 wherein said plurality of pouches contains a different said resin in each said pouch.

54. The method of harvesting a target protein of claim 46 wherein a plurality of said pouches is used.

55. The method of harvesting a target protein of claims 1 and claim 46 wherein said means of removing said protein-resin conjugate from said liquid comprise removing said pouch or pouches from said liquid.

56. The method of harvesting a target protein of claim 1 wherein said means of contacting said target protein with said resin comprise adding to said liquid a solid surface impregnated with said resin.

57. The method of harvesting a target protein of claim 56 wherein said solid surface comprises a two-dimensional or a three-dimensional structure.

58. The method of harvesting a target protein of claim 56 wherein said solid surface is a flat sheet, a plate, a plurality of plates connected together, a honeycomb, a wafer or any combination thereof.

59. The method of harvesting a target protein of claim 1 wherein said resin is the resin used to purify said protein in a downstream process.

60. The method of harvesting a target protein of claim 59 wherein said resin-protein conjugate is loaded in a downstream purification column and said protein purified.

61. A method of purification of a recombinant protein in a culture media with host cells present at the end of a bioreaction cycle, said method comprising:
Adding to said culture media a sufficient quantity of a resin suitable for the first stage of purification of said recombinant protein in a quantity capable of binding into a resin-protein conjugate, all or substantially all of said protein present in said culture media;
Transferring said settled resin-protein conjugate to downstream purification column;
Continuing with pre-established purification protocols of said purification column.

62. The method of purification of claim 61 wherein said resin-protein conjugate is allowed to settle in a container holding said culture media and then transferring said settled resin-protein conjugate through a drain port in said first container to said purification column.

63. The method of purification of claim 61 wherein said resin-protein conjugate is allowed to settle down, and said liquid decanted, and said settled resin-protein conjugate transferred to said downstream purification column.

64. The method of purification of claim 61 wherein said resin is first packaged in a pouch made of a filter material capable of retaining said resin within said pouch, allowing sufficient time for the said resin-protein conjugate to form, removing said pouch containing resin-protein conjugate and transferring it to said downstream purification column without removing said resin-protein from said pouch.

65. The method of purification of claim 61 wherein said resin is first packaged in a pouch made of a filter material capable of retaining said resin within said pouch, allowing sufficient time for the said resin-protein conjugate to form, removing said pouch containing resin-protein conjugate, opening said pouches and transferring said resin-protein conjugate to said downstream purification column.

66. The method of harvesting a target protein of claim 46 wherein said pouch is pre-installed inside said container prior to starting a reaction and filled with said resin when the solution is ready for harvesting.

67. The method of harvesting a target protein of claim 1 wherein the quantity of said resin used is determined by first analyzing the concentration of said target protein in said culture medium and matching it with the known binding capacity of said resin to said target protein.

68. The method of harvesting a target protein of claim 1 wherein said resin is added to said liquid in additional quantitates until such time that the concentration of said target protein in said liquid is less than 1% of the initial concentration of said target protein in liquid.
